(19) United States
(12) Patent Application Publication
(10) Pub. No.: US 2010/0076566 A1
(43) Pub. Date: Mar. 25, 2010

(54) Metal/alloy coated ceramic

(75) Inventors: Louis A. Serafin, JR., Lakeport, MI (US); Lee Allen Stouse, Brighton, MI (US); Leo A. Whiteside, Chesterfield, MO (US); Nicholas H. Burlingame, Belmont, NY (US)

Correspondence Address: CHRISTOPHER JOHN RUDY, 209 HURON AVENUE, PORT HURON, MI, 48060, US

(73) Assignee: SIGNAL MEDICAL CORPORATION, Marysville, MI (US)

(21) Appl. No.: 12/584,482

(22) Filed: Sep. 4, 2009

Related U.S. Application Data
(63) Continuation-in-part of application No. PCT/US2008/002768, filed on Mar. 1, 2008.
(60) Provisional application No. 60/904,803, filed on Mar. 5, 2007.
(60) Provisional application No. 60/930,157, filed on May 14, 2007.
(60) Provisional application No. 60/930,862, filed on May 18, 2007.

(51) Int. Cl. A61F 2/38; B32B 15/04; B32B 3/26

(52) U.S. Cl. 623/20.32; 428/469; 428/319.1

(57) ABSTRACT

Ceramic surface can be coated with a metal or metal alloy and have extraordinary holding power between the surface and coating. For example, MgTTZ can be coated with CPT and may have a static shear strength for the coated surface of at least about 2,000 or 3,000 or 5,000 or 7,000 pounds or greater. The coated ceramic may be a prosthetic surgical load bearing implant.

This continues in part, i.e., is a continuation-in-part of, international patent application No. PCT/US2008/002768 filed on Mar. 1, 2008 A.D., which, as does the present application, claims priority benefits under the Patent Cooperation Treaty and/or Title 35 United States Code, particularly under sections 119(e), 120, 363 and/or 365, of U.S. provisional patent application Nos. 60/904,803 filed on Mar. 5, 2007 A.D., 60/930,157 filed on May 14, 2007 A.D., and 60/930,862 filed on May 18, 2007 A.D. The specifications of those applications in their entireties, which of course include their drawings, are incorporated herein by reference.

### FIELD AND PURVIEW OF THE INVENTION

This concerns a ceramic coated with a metal or metal alloy. In one embodiment, it is a surgical implant, which can be a prosthetic load-bearing implant, say, made with a zirconia ceramic having the metal or metal alloy porous coating. For example, the implant or component can embrace femoral and/or tibial component(s) for a human knee, say, made of a partially stabilized zirconia (PSZ) such as a magnesium oxide stabilized transformation toughened zirconia (MgTTZ) coated with a commercially pure Titanium (CPT) coating.

### BACKGROUND TO THE INVENTION

Certain metal or metal alloy porous coated ceramic materials such as prosthetic implants are known. These can be intended for load bearing.

Chamier et al., U.S. Pat. No. 6,319,285 B2, discloses a ceramic acetabular cup with metal coating, for example, as a 6-4-1 ELI alloy coated alumina acetabular cup. Drawbacks with such a disclosure, however, include narrow applicability and strength of coating to ceramic that can be less than desirable.

Goodman et al., U.S. Pat. No. 5,766,257, discloses an artificial joint having natural load transfer. In a particular embodiment, it is a knee. Preferably the joint is of metal construction with an ultra high molecular weight polyethylene (UHMWPE) tibial tray liner. Additional modularity may be provided in such an implant. See, Serafin, Jr., U.S. Pat. No. 6,629,999 B1. Such load bearing implants and more can be made of ceramic, and, in particular, a PSZ ceramic, for example, MgTTZ. See, Serafin, Jr., et al., patent application Pub. No. US 2006/0025866 A1 for ceramic manufactures. See also, Serafin, Jr., et al., WO 2004/0830340 A2 and A3. The '866 and '340 publications disclose that an interiorly facing bone ingrowth enhancing surface may be provided the ceramic frame through coating by tantalum vapor deposition techniques known in the art.

Serafin, Jr., et al., U.S. patent application Ser. No. 11/657,385, discloses a metal/alloy on ceramic coating. It has a more broad applicability. An exemplary ceramic is MgTTZ, and exemplary physical attachment features include undercut grooves and/or holes.

Also, certain metal components may have ceramic coatings. Thus, some metal jet engine parts may have ceramic coatings, and Bekki et al., U.S. Pat. No. 5,007,932, discloses an artificial bone joint, which may employ metal to form the body, and the surface may be coated with ceramics.

The foregoing cited art, including drawings, is incorporated herein by reference.

It would be desirable to ameliorate if not overcome problems or drawbacks in the art. It would be desirable to improve the art and/or provide alternatives to it.

### A FULL DISCLOSURE OF THE INVENTION

In general, the present invention provides a coated ceramic comprising a fired ceramic body having a surface; and, on at least part of the surface, the metal/metal alloy coating. The porous coating can be from a metal or metal alloy other than by tantalum vapor deposition. The coated ceramic can be an orthopedic implant or component for an orthopedic implant where the fired ceramic body comprises a body for the implant or component, which has a surface; and, on at least part of the surface, the metal/metal alloy coating. The orthopedic implant or component can be a load bearing implant or component for a load bearing implant having an articular surface and a nonarticular surface, where the porous coating is on at least part of the nonarticular surface. A method to provide the coated ceramic is provided.

The invention is useful in providing metal/metal alloy coatings to ceramic manufactures. It can be useful as an implant in the field of orthopedics.

Significantly, by the invention, the art is advanced in kind. Not only is a ceramic body coated with a metal or metal alloy, but extraordinary holding power of the coating to the ceramic can be provided. Thus, for instance, zirconia ceramic prosthetic implants or implant components, especially, for example, those made with an MgTTZ ceramic, and notably those which are load bearing, can be provided with a reliable Titanium or Titanium alloy porous coating, for example, CPT, which may be subjected to nitric acid treatment for forming a nitride; and so, the surgeon can have a most dependable implant system, which, in addition to its dependability, can ameliorate if not avoid allergic reaction to standard metal implants in certain patients such as, for example, patients allergic to the Nickel of a conventional Cobalt-Chrome femoral component for a knee. The invention is efficient in manufacture as well. Numerous further advantages attend the invention.

The drawings form part of the specification hereof. With respect to the drawings, which are not necessarily drawn to scale, the following is briefly noted:

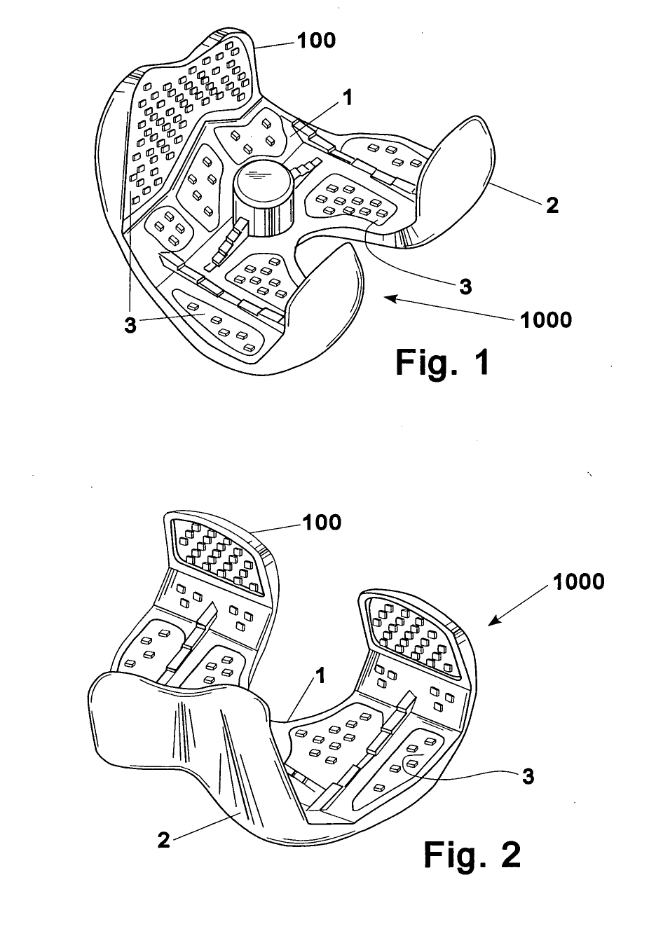

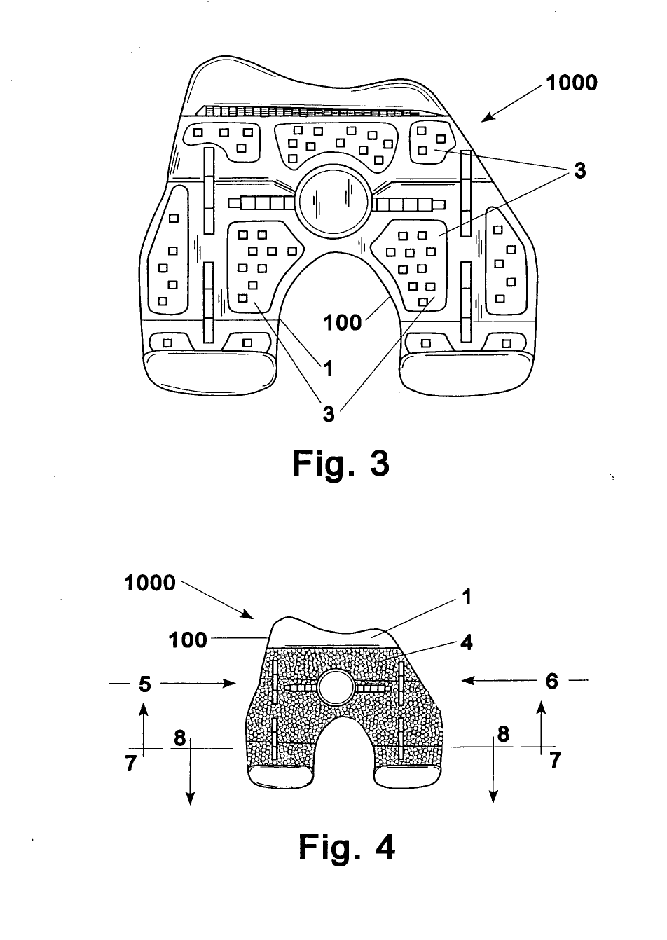

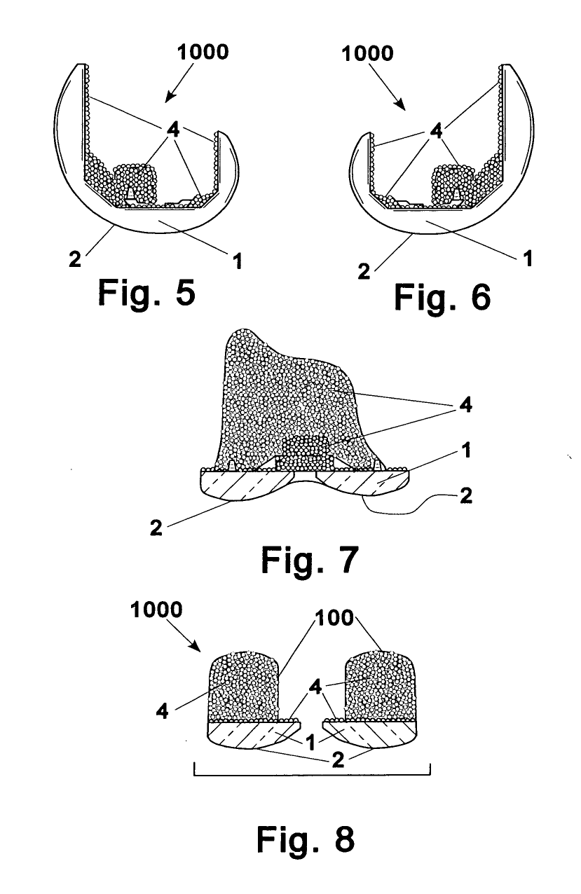

FIGS. 1-8 depict a femoral implant component for a human cruciate-retaining knee joint implant, a type of a component for a non-rotating hinge knee implant, for example, made with MgTTZ. FIGS. 1-3 are views of the component without coating, with FIG. 1 a rear, top perspective view; FIG. 2 a front, top perspective view; and FIG. 3 a top view. FIGS. 4-8 are views are of the ceramic component of FIGS. 1-3 coated, for example, with a CPT porous coating, with FIG. 4 a top view; FIG. 5 a side view taken along arrow 5 in FIG. 4; FIG. 6 a side view taken along arrow 6 in FIG. 4; FIG. 7 a sectional view taken in the direction of line 7-7 in FIG. 4; and FIG. 8 a sectional view taken in the direction of line 8-8 in FIG. 4.

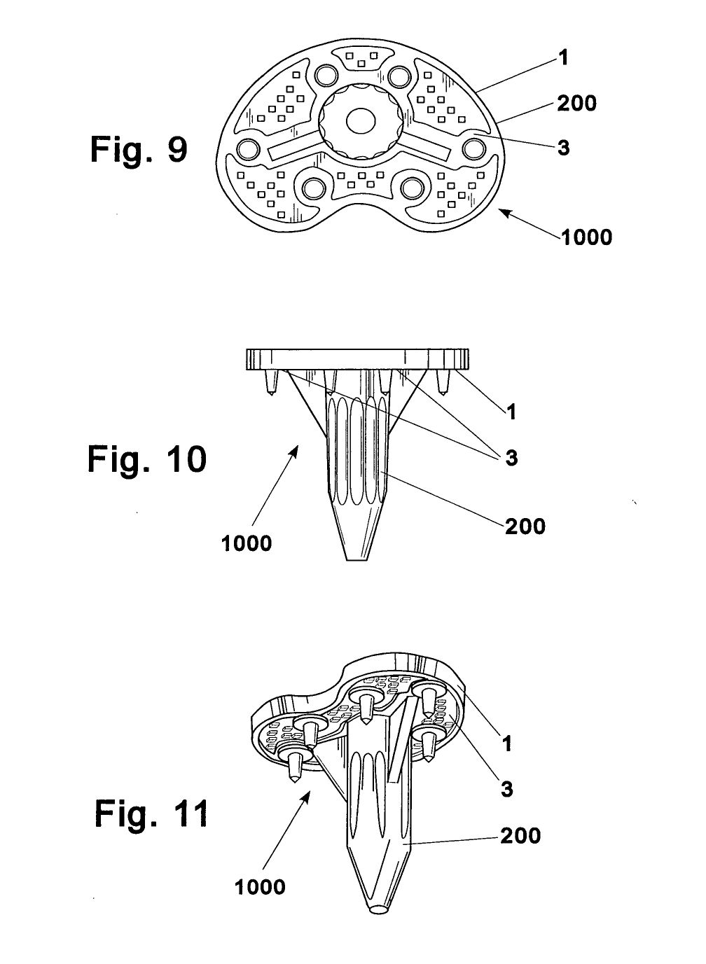

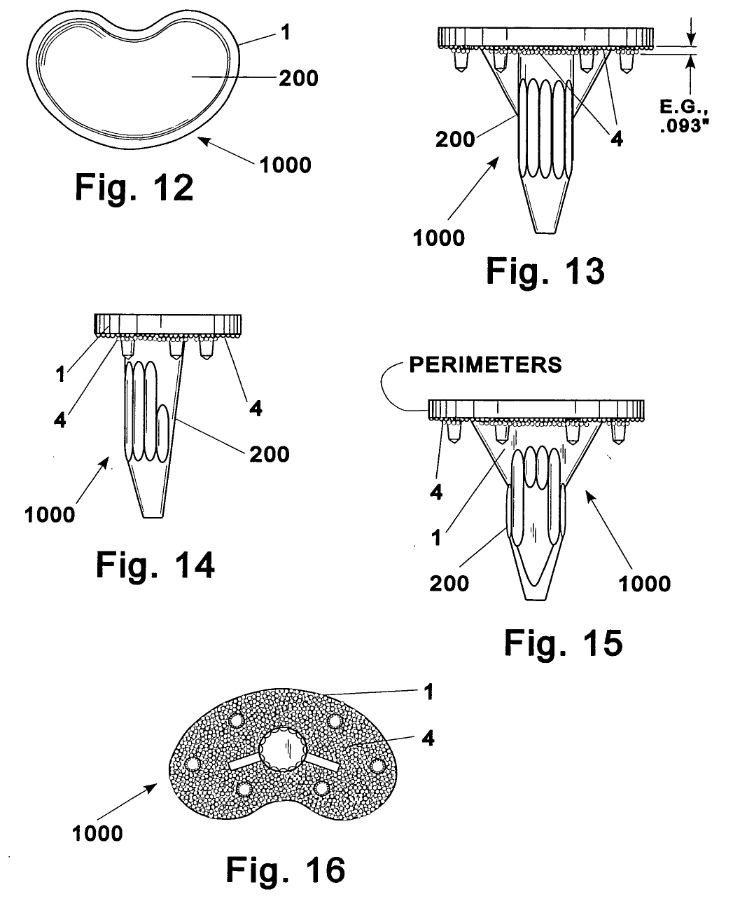

FIGS. 9-16 depict a tibial tray implant component for a tibial portion of a human knee joint, for example, made with MgTTZ. FIGS. 9-11 are views are of the component without coating, with FIG. 9 a bottom view; FIG. 10 a side view; and FIG. 11 a bottom perspective view. FIGS. 12-16 are views are of the ceramic component of FIGS. 9-11 coated, for example, with a CPT porous coating, with FIG. 12 a top view; FIG. 13 a side view in elevation; FIG. 14 a side view in elevation at a 90-degree angle to that of FIG. 13; FIG. 15 a side view in elevation at a 180-degree angle to that of FIG. 13; and FIG. 16 a bottom view.

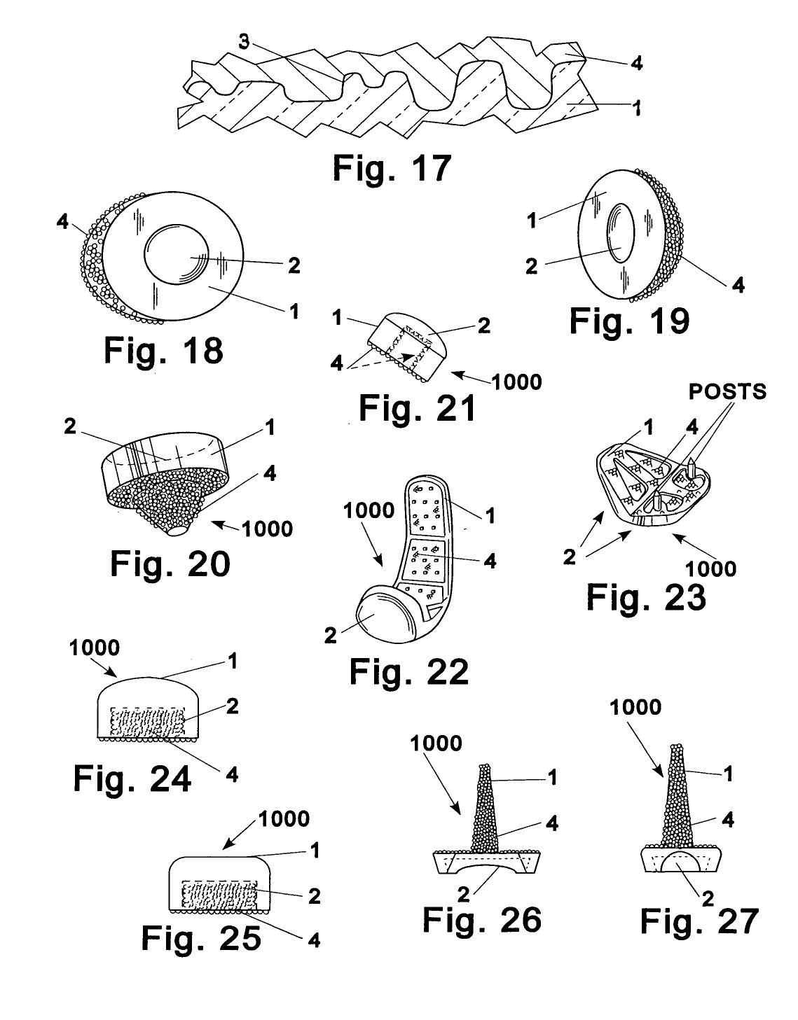

FIG. 17 is a sectional view of a proposed ceramic-metal/metal alloy interface.

FIGS. 18-52 depict other exemplary embodiments of the invention, to wit:

FIGS. 18 and 19 are perspective views of cups for some enarthrodial joint implants, with FIG. 18 an acetabular cup; and FIG. 19 a glenoid cup.

FIG. 20 is top plan view of a great toe implant.

FIG. 21 is a side plan view of a temporal mandibular joint

FIG. 22 is a rear, perspective view of a unicompartmental femoral component condylar implant

FIG. 23 is a “top,” perspective view of a patellofemoral implant.

FIGS. 24-27 are views of an ankle implant or ensemble, with FIG. 24 a side plan view of a talus cap, which may be employed by itself as a hemi-implant; FIG. 25 a front plan view of the talus cap of FIG. 24; FIG. 26 a side plan view of a tibial tray that may be employed with a talus cap as of FIG. 24; and FIG. 27 a front plan view of the tibial tray of FIG. 26.

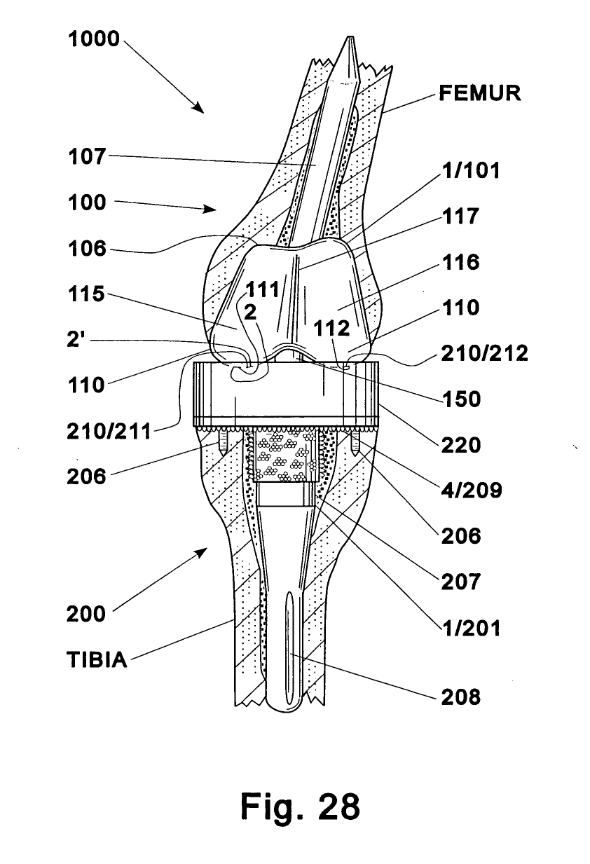

FIG. 28 is a front view of an embodiment of a rotational knee joint implant that may have at least a ceramic component body among its femoral and tibial components, at least a portion of which ceramic body is metal/alloy porous coated, and which has a rotation device.

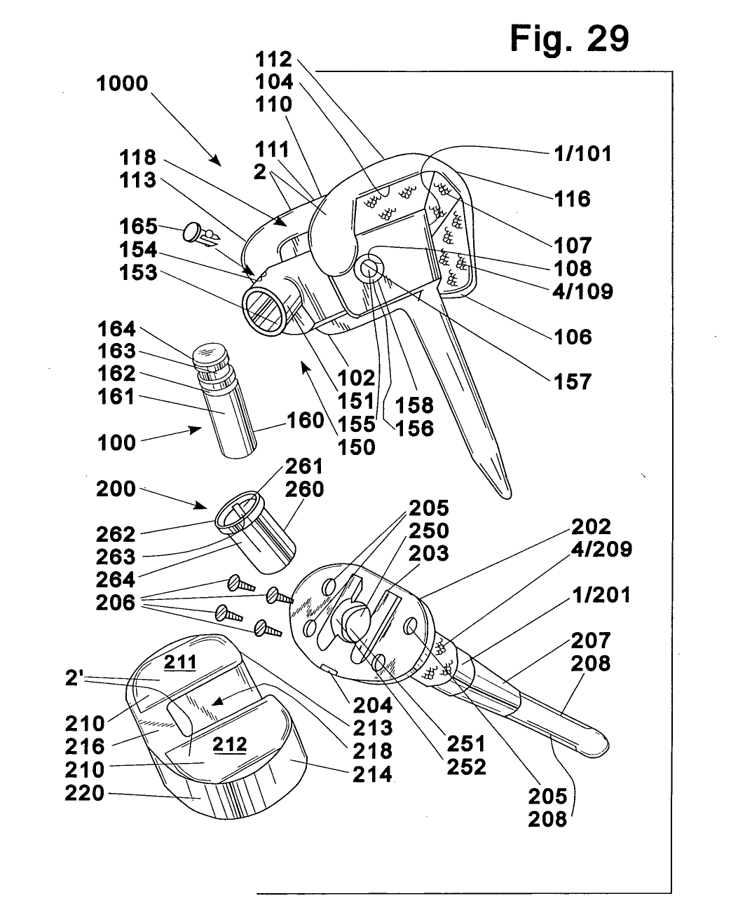

FIG. 29 is an exploded view of the joint implant of FIG. 29.

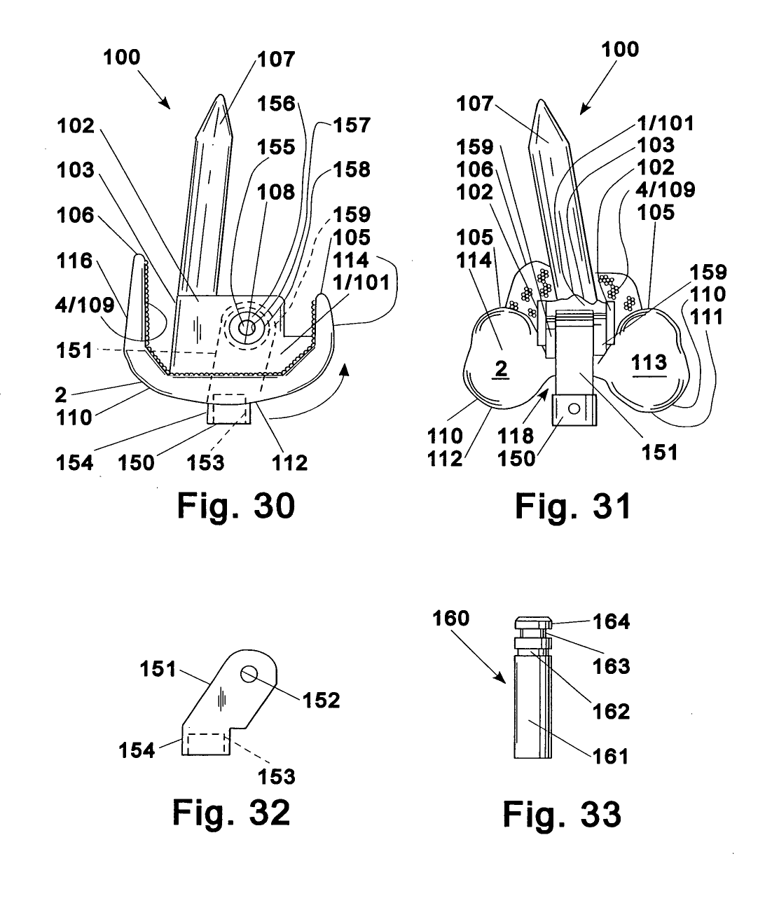

FIG. 30 is a left side view of the femoral component to the joint of FIG. 28.

FIG. 31 is a rear view of the femoral component of FIG. 30.

FIG. 32 is a left side view of the rotation device member of the femoral component seen in FIGS. 28-31.

FIG. 33 is a side view of the rotation device femoral-tibial taper pin of the joint implant as seen in FIG. 29.

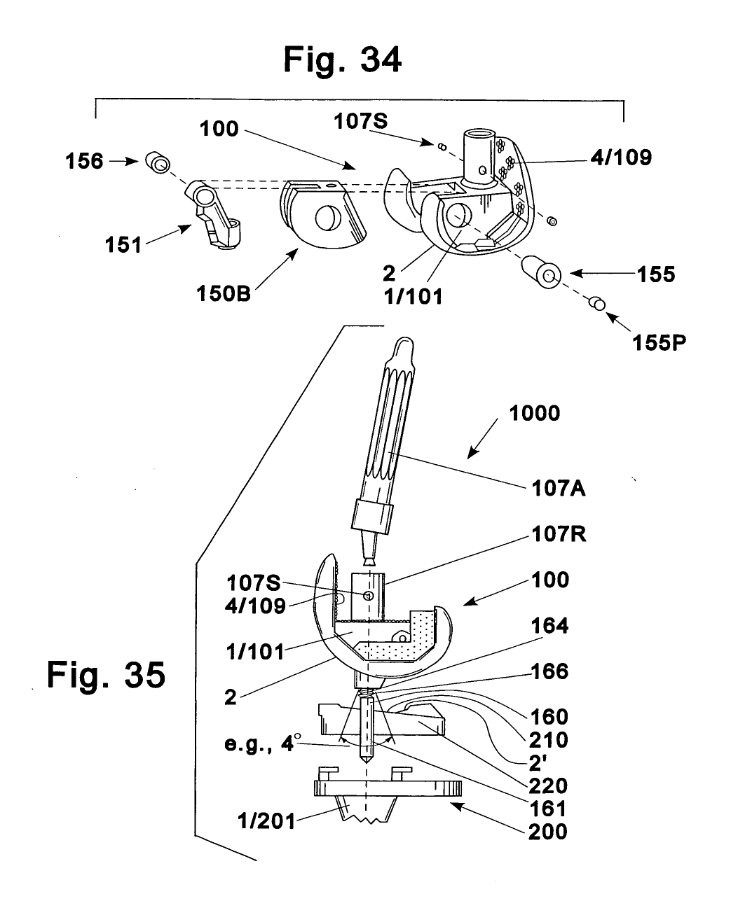

FIG. 34 is an exploded, perspective view of a femoral component of another artificial prosthetic rotational knee joint implant.

FIG. 35 is an exploded side view of the artificial prosthetic rotational knee joint implant having the femoral component of FIG. 34.

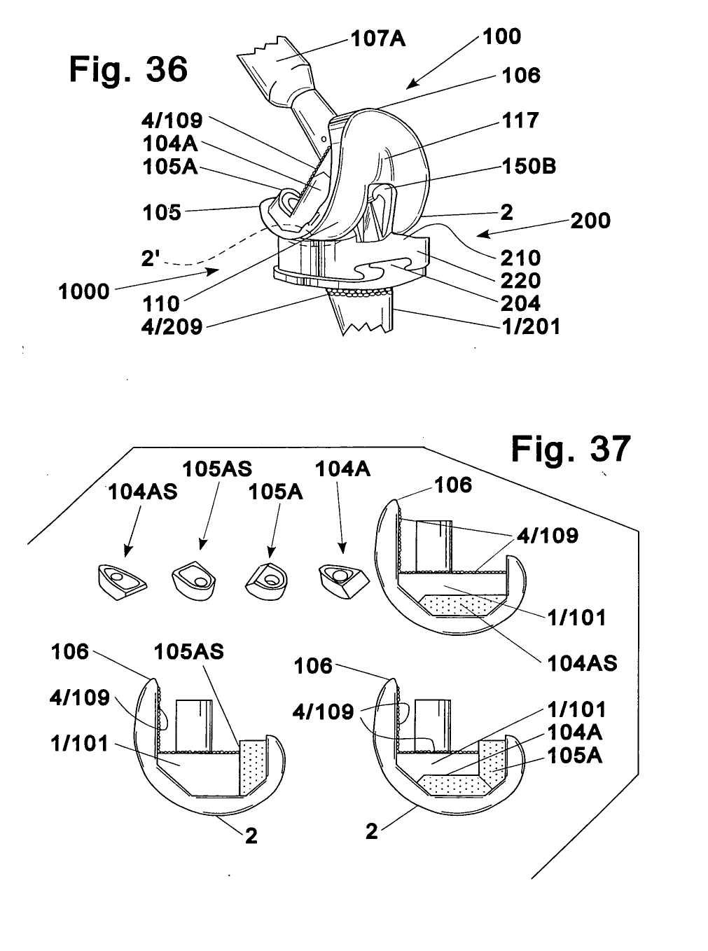

FIG. 36 is a front, perspective view of the implant of FIG. 35, assembled and having several augments in its femoral component to accommodate bone loss.

FIG. 37 shows perspective and side views illustrating various femoral augments, some of which can be seen within FIGS. 35 and 36.

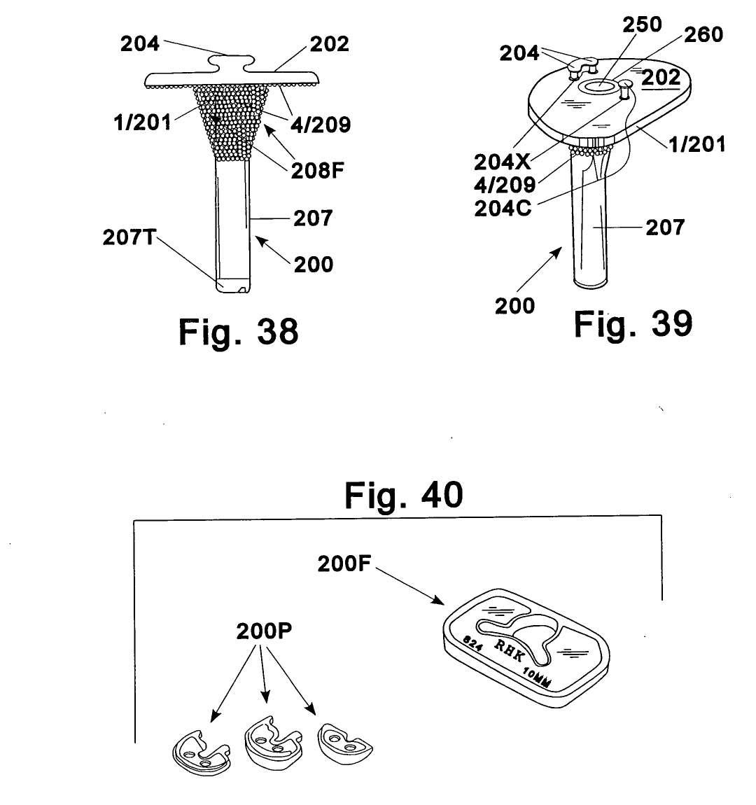

FIG. 38 is a side view of a tibial base plate in the implant of FIGS. 35 and 36.

FIG. 39 is a top, perspective view of the tibial base plate of FIG. 38.

FIG. 40 is a perspective view of some partial tibial augments, which may be employed with the tibial base plate of FIGS. 38 and 39.

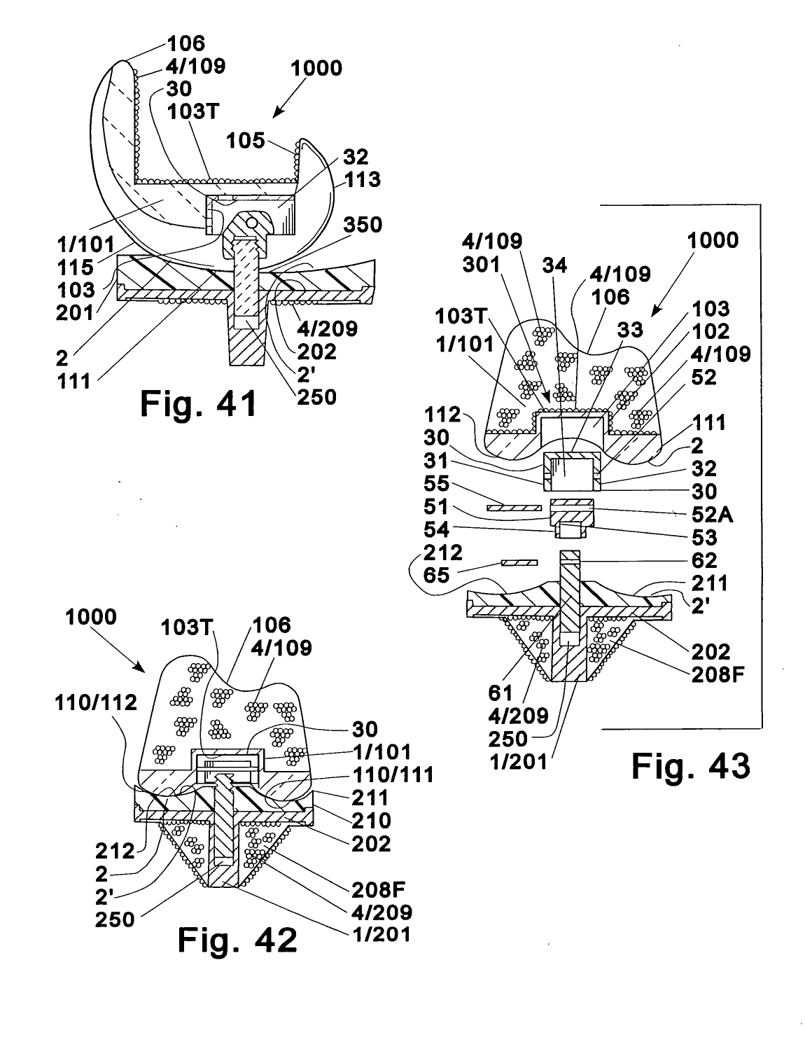

FIG. 41 is a saggital sectional view of a modular porous coated rotational knee joint implant.

FIG. 42 is a rear, sectional view of the implant of FIG. 41.

FIG. 43 is an exploded, rear sectional view of a modular implant, similar to that of FIGS. 41 and 42, employing pin type attaching of its axial (taper) pin.

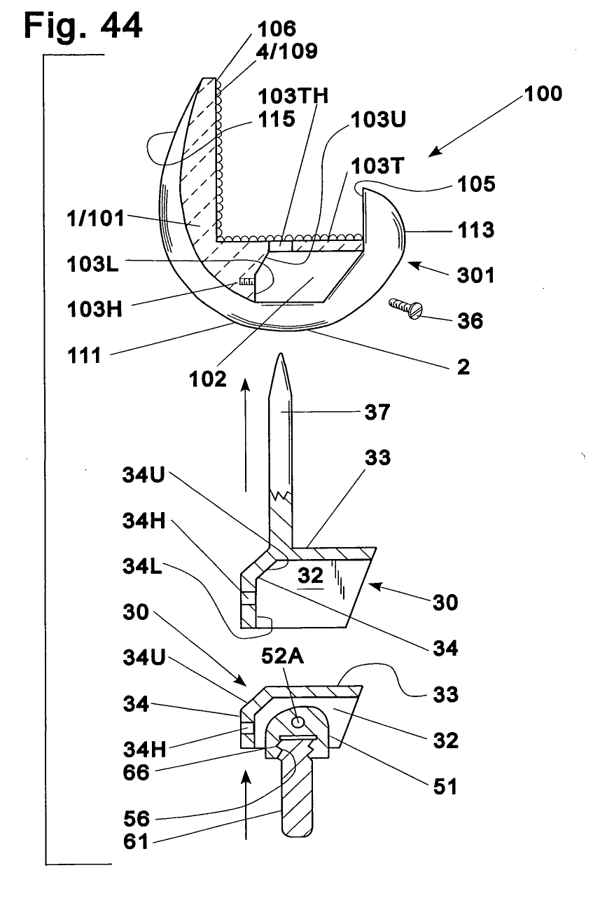

FIG. 44 is an exploded, saggital sectional view of a coated ceramic modular rotational knee joint implant, with module-in-module modularity.

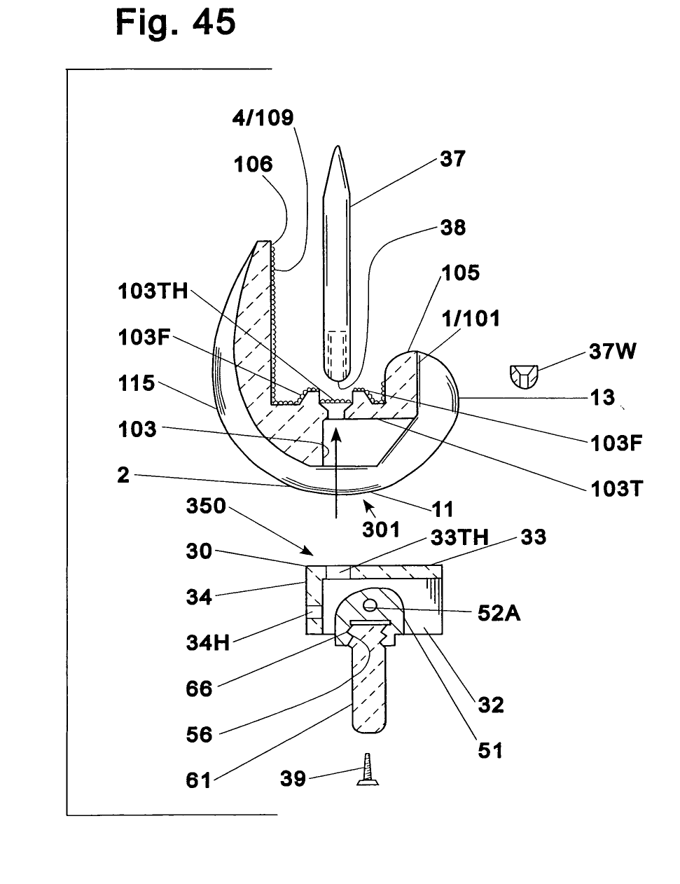

FIG. 45 is an exploded, saggital sectional view of a coated ceramic modular rotational knee joint implant, with a top-insert stem.

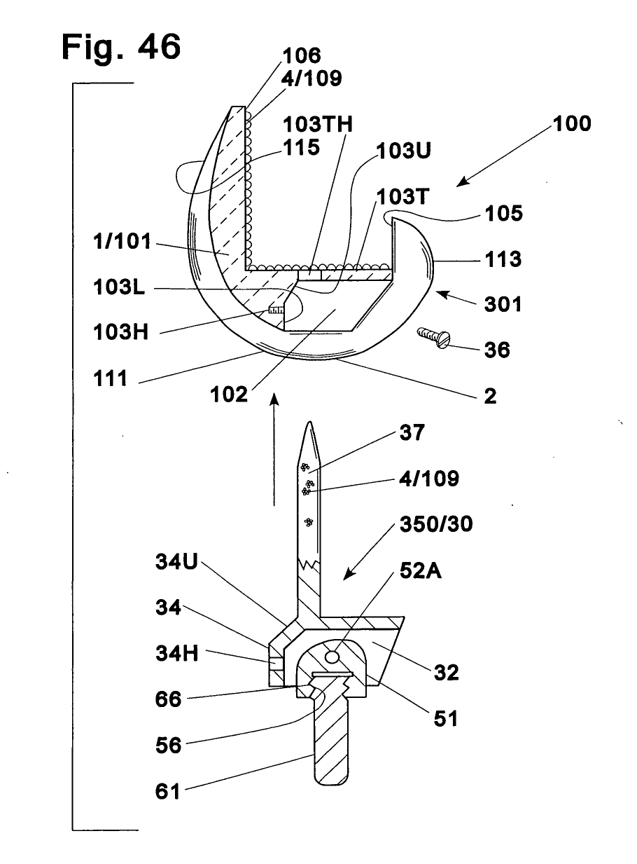

FIG. 46 is an exploded, saggital sectional view of a coated ceramic modular rotational knee joint implant, with a one-piece stem and box.

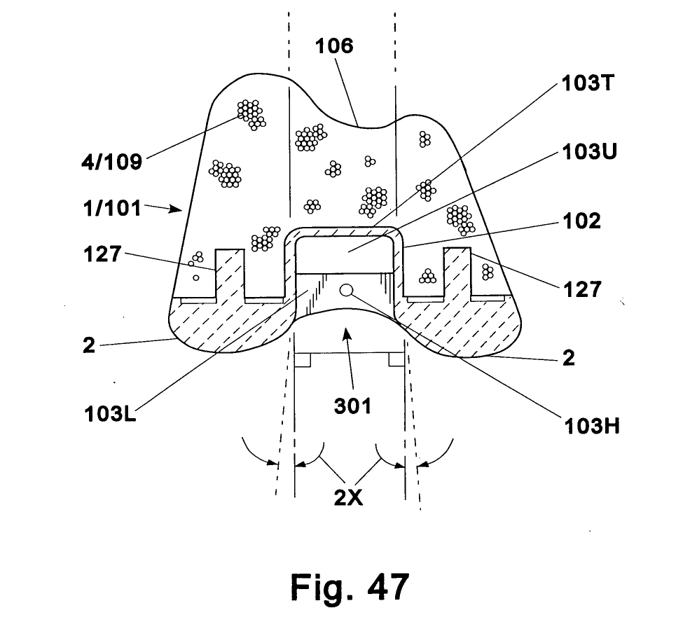

FIG. 47 is a rear view in section of the femoral component frame such as seen generally within FIGS. 44 and 45, or being a replacement therefor.

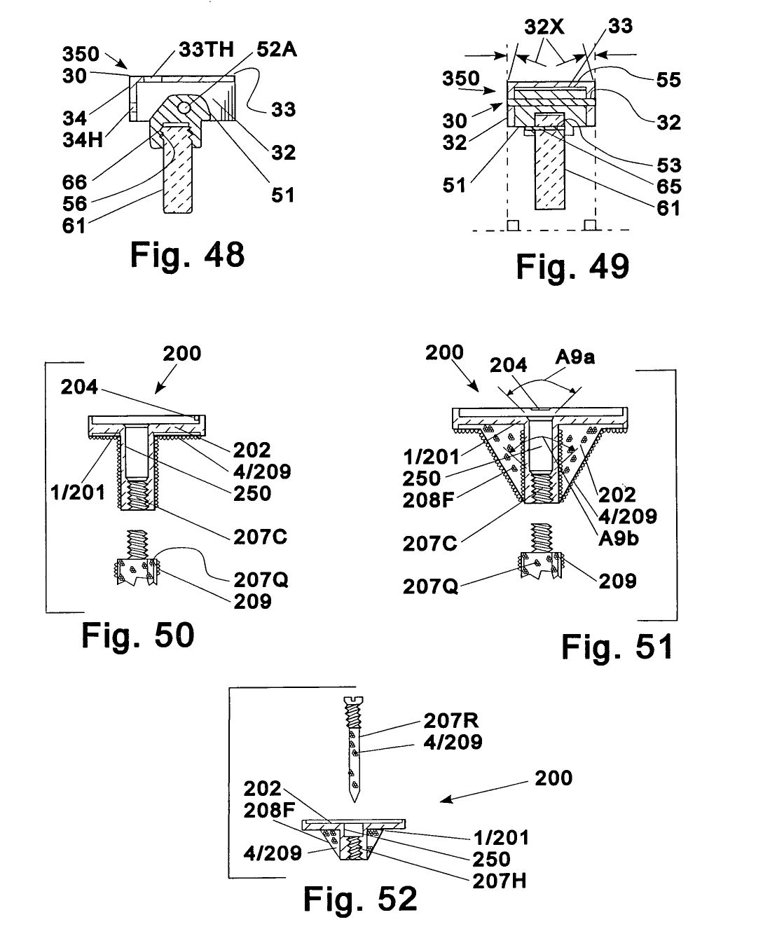

FIG. 48 is a saggital sectional view of the insertable rotation device with a swingable, depending male type part of the implant such as of FIGS. 41 and 43.

FIG. 49 is a rear sectional view of the insertable rotation device of FIG. 48.

FIG. 50 is an exploded side view of another embodiment of a modular porous coated ceramic tibial tray, which may be employed with a suitable femoral component having a depending male type rotating part.

FIG. 51 is an exploded rear view of the tray of FIG. 50.

FIG. 52 is an exploded rear view of another modular porous coated ceramic tibial tray, which also may be employed with a suitable femoral component.

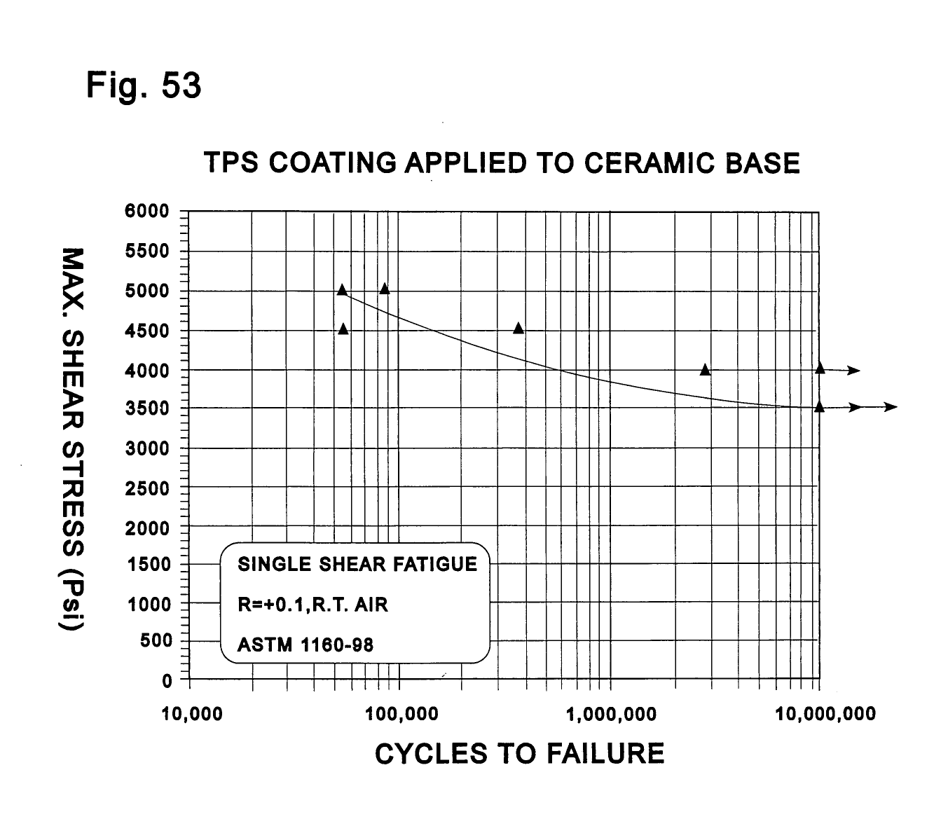

FIG. 53 is a graph of maximum shear stress versus cycles to failure for MgTTZ specimens with a CPT titanium plasma spray (TPS) porous coating.

The invention can be further understood by the detail set forth below, which may be read in view of the drawings. As with the foregoing, the following is to be understood in an illustrative and not necessarily limiting sense.

The present coated ceramic embraces a fired ceramic body, which has a surface and serves as a substrate. The same can be a surgically implantable implant or component thereof, which may be for a human patient. The implant can be intended for a load bearing application. Some, or in some cases perhaps all, of the surface may be roughened to accommodate a metal or metal alloy coating. On some, or in some cases perhaps all, of the roughened surface is provided with the metal or metal alloy coating. Other surface(s) of the coated ceramic can retain unroughened and/or uncoated ceramic surface(s), which, for example, in the case of a prosthetic implant, can include load-bearing, polished articulating surface(s). Such unroughened surfaces may be coated with the metal or metal alloy coating.

Any suitable ceramic can be employed. The ceramic can be a zirconia ceramic. In one embodiment, the ceramic is a PSZ ceramic such as an MgTTZ ceramic. See, e.g., the aforementioned patent application publications by Serafin et al., Pub. No. US 2006/0025866 A1 and WO 2004/0830340 A2 and A3. Accordingly, before application of the porous coating, the ceramic body can be prepared by a method as found therein or as otherwise known and/or practiced in the art. The MgTTZ ceramic can conform to ASTM F 2393-04, “Standard Specification for High-Purity Dense Magnesia Partially Stabilized Zirconia (Mg-PSZ) for Surgical Implant Applications.” Polishing of articular or other surface(s) can precede or follow roughening of the target surface and/or porous coating.

In one embodiment, the metal/metal alloy coating is provided on a roughened surface of the fired ceramic body. The roughened surface may be provided at any suitable time, to include, before and/or after firing, for example, after firing.

For instance, to obtain the roughened surface, a target surface of a fired ceramic body can be grit-blasted with particles of a substance harder than the hardness of the ceramic body about the target surface. Thus, as an illustration, alumina particles can be employed to blast an MgTTZ ceramic target surface to provide the roughened surface. For example, a bone-interface surface are of an MgTTZ prosthetic implant can be grit-blasted with alumina grit, which may include about from a 10-grit to a 20-grit or even a 25-grit or 30-grit particle size, say, about a 16-grit particle size, at a suitable pressure from a grit-blasting device, which may include about from a 50-psi to 100-psi (about from a 3.5-kg/cm² to 7-kg/cm²) pressure, say, about an 80-psi (about a 5.6-kg/cm²) pressure, for enough time to provide a roughened surface, which, independently at each occurrence, with an about from a 2-micron, 3-micron, or especially a 5-micron, 6-micron or 10-micron arithmetic average surface finish (2-Ra, 3-Ra, 5-Ra or 10-Ra) to about a 8-Ra, a 10-Ra, 13-Ra, 15-Ra or 20-Ra value as is known in the art. Thus, the ceramic Ra-value can be about from 6-Ra to 8-Ra, or about from 10-Ra to 13-Ra, say, about 12-Ra. Care may be taken in general to not impinge on the target surface for too long a time; otherwise the roughened surface can become “concave.”

However, such a roughened ceramic surface need not necessarily be employed. In other words a more smooth surface, for example, an about from 1-Ra to 2-Ra finish or more smooth surface finish, say, an about from 0.5-Ra to 1.5-Ra, to include an about 1-Ra, finish as may be found on a polished condyle of a femoral knee implant, may be employed and provided with the metal or metal alloy coating. In addition, even rougher surfaces than an about 20-Ra ceramic surface, for example, an unpolished ceramic surface, say, an outer surface on a tibial tray liner, which may have an about from 30-Ra or 40-Ra to 60-Ra or 75-Ra, which would include an about 50-Ra ceramic surface, may be employed and provided with the metal or metal alloy coating.

It is contemplated that angle, distance, velocity, density and/or heat of spray may play a significant part in forming the ceramic coated with the metal or metal alloy. Other factors may play a part. Among these can be the materials employed as the substrate and coating.

Any suitable metal or metal alloy can be employed as the coating. The coating may be Titanium metal or an alloy with Titanium. The coating can be CPT. Note, ASTM F-67.

For example, CPT can be sprayed by a plasma arc under vacuum, but with the metal powder being carried by Argon pick-up gas through a robotic sprayer, to be met with a flow of Hydrogen gas to enhance the heat of the spray, which mixture is carried through the plasma arc of the sprayer, and onto the roughened surface that is to be coated. The sprayer also can deliver Argon gas, say, from jet spray openings spaced laterally from the central spray with the metal, so that the Argon is directed to the surface so as to cool the ceramic as soon as the liquid metal hits the ceramic.

The metal or metal alloy coating can be provided to any suitable extent or thickness. As an illustration, in an orthopedic implant, for example, femoral and tibial tray components to a human knee joint implant, or to the outside of cups of an enarthrodial joint implant, a CPT porous coating can be applied to a thickness about from 0.015 to 0.025 of an inch (about from 0.038 to 0.064 cm) with an about 100-micron to about 300-micron pore size.

The metal/alloy coating may be applied to the ceramic in layers. A thinner initial coating layer may be applied to the ceramic, and then optionally cooled, before applying subsequent layer(s) of the metal/alloy coating.

The metal/alloy coating may be applied as one substantially uniform sample of metal or alloy. It may be applied as two or more samples of metal or alloy, say, by varying the metal/alloy composition during uninterrupted application or by providing the metal or alloy as differing layers.

Extraordinary holding power of the coating to the ceramic can be provided. For instance, the metal or metal alloy coating may resist being pulled or sheared off the ceramic to a value of about 2,000 pounds (about 0.91 metric tons) or more of force, to include about 3,000 pounds (about 1.4 metric tons) or more of force, or about 4,000 pounds (about 1.8 metric tons) or more of force, or about 5,000; 6,000; 7,000; 8,000; 9,000 or even 10,000 pounds (about 2.3; 2.7; 3.2; 3.6; 4.1 or even 4.5 metric tons) or more of force. Compare, ASTM F-1044-05, ASTM F-1160-98 and ASTM F-1659-95. Many of such values meet or exceed United States Food and Drug Administration (USFDA) requirements for metal or metal alloy porous coatings on metal or metal alloy implants.

With more particular reference to the drawings, metal and/or metal alloy coated ceramic implant 1000 can be embodied as a load bearing prosthetic implant or component therefor. The implant 1000 may be made, say, of MgTTZ with a TPS CPT porous coating, and it may be modular.

The implant 1000 generally has ceramic body 1, articular surface 2, receiving surface 3 for receiving the metal or metal alloy coating, which may be roughened or not roughened, and metal/metal alloy porous coating 4. The articular surface 2 is generally smooth, and may be polished, as part of the ceramic body. In other words, the articular surface can be generally provided as a smooth ceramic surface of the ceramic body 1. However, an articular surface may be of a material other than the ceramic body 1 such as by being a coated metal or metal alloy on the ceramic body, which coated metal or alloy on the ceramic body substrate is made to be smooth and suitable for the articulation under consideration. An articular surface 2′ may also be made as an insert that may be attached to the ceramic body such as being a polyethylene insert as a liner for a metal/metal alloy coated ceramic tibial tray, or as a liner with dovetail ridge(s) that slide into corresponding undercut groove(s) in an appropriate surface of the ceramic body. Depending on the configuration and application of the implant 1000 with its ceramic body 1 and articular surface 2, 2′, in general, the receiving surface 3 can be provided at, and the metal/metal alloy porous coating 4 can be applied on the receiving surface 3 to, any suitable location of the ceramic body 1. Thus, for example, after grit blasting all inner surfaces of “box” geometry of a ceramic femoral knee implant component 100, say, of MgTTZ, say, with 16-grit aluminum oxide, to provide a roughened receiving surface 3, the metal/metal alloy porous coating 4, say, CPT, may be applied thereto, say, by TPS, to a thickness of about from 0.015 to 0.025 inch (about from 0.038 to 0.064 cm). Or, after grit blasting the complete under area of a ceramic tibial tray of a knee joint tibial component 200, say, of MgTTZ, as well as any upper inset, say, with 24-grit aluminum oxide, without grit blasting around the perimeter of the tibial tray, to provide a roughened receiving surface 3, the metal/metal alloy porous coating 4, say, CPT, may be applied, say, by TPS, to a thickness of about from 0.015 to 0.025 inch (about from 0.038 to 0.064 cm). Likewise with other embodiments, a suitable area of the ceramic body 1 onto which the metal/metal alloy coating 4 is to reside, at least in part, may be roughened, left not roughened, i.e., as is, or perhaps even polished, to provide the receiving surface 3, and then the metal/metal alloy coating 4 applied thereon.

The implant 1000 can be a metal or metal alloy porous coated ceramic rotational knee joint implant or component therefor or for another suitably corresponding knee joint implant ensemble. The same may be modular, and the following is noted more particularly with respect to such embodiments:

Femoral component 100 can include femoral component frame 1/101, which may be of a one-piece ceramic construction. The frame 101 can include side walls 102; front wall 103, which may have upper segment 103U, lower segment 103L and/or hole 103H that may be tapped to receive screw 36; and top 103T, which may have hole 103TH and may have supporting flange 103F, which may accommodate inferiorly insertable intramedullary femoral spike 37. The spike 37 may be part of a boxlike module 30 that includes side walls 32, front wall 34 that may have upper portion 34U and lower portion 34L, and top 33, which mate closely with the walls 102, 103, 103U, 103L and the top 103T; and/or that includes hole 34H through which the screw 36 may pass on its way to the hole 103H. The spike 37 may be secured with washer 37W, and have screw-receiving hole 38 threaded for receiving screw 39 that also secures boxlike modular rotation device 350. The frame 1/101 can also include distal condylar flange 104; posterior flange 105; anterior flange 106; femoral bone stock insertion stem 107, which may be separately addable stem 107A to stem receptacle 107R and be secured by set screw 1075; and wall hole for integral rotation device 150. Femoral bone loss augments 104A, 105A for use together, and 104AS, 105AS for separate use, may be provided, for example, of ceramic or other suitable material, which may be porous coated by metal and/or metal alloy. Interiorly facing bone-ingrowth metal/alloy porous coating 4/109 can be provided, for example, by plasma spray, over suitable target ceramic surface. The target surface may be roughened by grit-blasting or not. Femoral condylar articular surface 2/110 of generally convex geometry generally includes inferior, medial condyle 111; inferior, lateral condyle 112; posterior, medial condyle 113; posterior, lateral condyle 114; and may be considered to include anterior, medial condyle 115 and anterior, lateral condyle 116. On a superficial side of the anterior flange 106 can be provided trochlear surface 117, on which a natural or artificial knee cap may generally ride. Intracondylar notch 118, or inferiorly insertable module housing 301 for insertion of a modular rotation device 350 and/or modular spike 30/37, may be formed. The condylar surfaces 110-117 are articular surfaces in general, and they can be polished to a smooth micro-finish, which can be carried out before application of the porous coating 109. Condyle-backing femoral spikes 127 may be provided. The rotation device 150 or 350 is provided.

The rotation device 150, which may be substantially ceramic or may be metal in general, may include UHMWPE box insert 150B, and includes rotation member 151, generally with rotation member hole 152; taper pin receptacle 153, advantageously formed with a Morse taper-accommodating cup; and punch-pin hole 154. Axle 155, which may be secured by axle plug 155P, runs through the hole 152 and may run through radial bushing 156, say, of UHMWPE, which bushing has axle hole 157; insert shoulder 158, which fits snugly in the wall hole 108; and member-spacing shoulder 159. The rotation device 150 has highly polished taper pin 160, which can include cylindrical shaft 161; and may include extraction groove 162 to extract the pin 160 from the receptacle 153, say, with a prying tool during surgical implantation of the prosthesis 1000; extraction-restriction punch-pin locking groove 163; and taper lock tip 164, which can be made with a Morse taper to fix the pin 160 in the cup 153. When the pin 160 is so fixed, it may be set by insertion and fit of an extraction-restriction and/or rotation-restriction punch-pin 165 through the hole 154 and into the groove 163. Threads 166 may be present, preferably in conjunction with a Morse taper as the taper 164, as an alternative for fastening a modular taper pin 160.

The rotation device 350 is completely modular and inferiorly insertable into the insertable modular housing 301, preferably adapted for such with its walls 102 having a Browne & Sharpe taper 2X, or similar housing such as provided by the boxlike module with the spike 37, as an embodiment of the addable component 30, and can include swingable, depending male type part in housing 31 with side walls 32, preferably with a restraining Browne & Sharpe taper 32X; optional top wall 33, which may have top hole 33TH; and front wall 34. Holes 52 in the side walls 52 accommodate hinge pin (axle) 55. Pivot block (rotation member) 51 can have hole 52A, which continues along the direction of the holes 52; taper pin cup 53, which may be smooth walled and tapered, say, with a Morse taper, and/or provided with threads 56; and punch-pin hole 54. The taper pin 61 is inserted in the cup 53, and may be secured through punch pin 65 and/or threads 66. The rotation device 350 may be made with a one-piece depending male type part such as by having the components 51 and 61 made of one, integral piece.

The tibial component 200 can include tibial component frame 1/201, which can be generally made of ceramic or metal, and have tibial tray 202; dovetail liner insertion rails 203; liner-stopping ramp or rotation safety stop 204, and central stop 204C, particularly if part of double-capture locking mechanism 204X; screw holes 205 through which can be inserted bone-fastening screws 206; stem 207—which may be insertable inferiorly into receiving cup 207C that may be threaded, by provision of separate stem 207Q that may be threaded also; or which may be insertable superiorly, even after implantation of the component frame 201, through hole 207H that may be threaded, by provision of separate stem 207Q that has a superior screwing head with superior threads—and which may have distal taper 207T, a number of, say, three, distal ribbed grooves 208 and/or a number, say, two, underside flanges 208F; and an interiorly facing bone-ingrowth enhancing surface such as of metal/alloy porous coating 4/209, for example, by plasma spray, over suitable target ceramic surface if the frame 201 is made of ceramic. (A metal or metal alloy frame 201 may also be porous coated with metal and/or metal alloy.) The target surface may be roughened by grit-blasting or not. The tibial articular surface 2′/210 is of concave geometry in suitable complimentarity to the convex geometry of the condylar surface 2/110, and generally includes superior, medial articular surface 211 and superior, lateral articular surface 212 on medial lobe 213 and lateral lobe 214, respectively. On the underside of each lobe may be dovetail grooves 215 for sliding along any rails 203; lobe-spanning portion 216; notch 216 for locking in cooperation with the stop(s) 204, 204C; and intra-condylar notch 218 analogous to the notch 118. Ramp 219 may make for easier installment over the stop 204. Such features 201-219 may be provided with separable tibial tray liner 220 of suitable material, say, UHMWPE. Rotation device receptacle 250 may be in a form of an essentially cylindrical cup 251, which may have top shoulder recess 252. Rotation device receptacle liner 260, say, of UHMWPE, may be inserted in the receptacle 250 and its cup 251 so as to itself receive the taper pin 60, 160. The liner 260 can include taper pin accommodating cup 261; shoulder 262, which can fit in the recess 252; a number of, say, two to four, inside, axially directed grooves 263 to permit exit of entrained body fluids during extension and flexion of the implanted joint 1000 and consequent up and down motion of the taper pin 60, 160, which fits quite closely although movable within the liner cup 261; and outside axially directed fluid-escape feature 264, say, groove, or possibly hole, to permit escape of liquids and/or gasses during insertion of the liner 260 into the receptacle 250, between which there is a close, essentially immovable-in-use fit. Shoulder bevel angles A9a and A9b may be, say, respectively, 90° and 118° of angle.

Tibial block augments may be provided, for instance, full augment 200F or partial augment 200P. RHK full tibial block augments 200A can only be used with RHK tibial base plates. The table, which follows, lists some augments available from Zimmer, Inc.

| Tibial | | RHK Full | NexGen Partial |
|---|---|---|---|
| Size | M-L × A-P | Augments | Augments |
| 1 | 58 × 41 mm | Size 1 | Size 1 |
| 2 | 62 × 41 mm | Size 2 | Size 2 |
| 3 | 67 × 46 mm | Size 3 | Size 4 |
| 4 | 70 × 46 mm | Size 4 | Size 4 |
| 5 | 74 × 50 mm | Size 5 | Size 6 |
| 6 | 77 × 50 mm | Size 6 | Size 6 |

Beneficially, the knee implant 1000 has natural load transfer. As such, the knee implant may carry a substantial amount, say, about ninety percent or more, or about ninety-five percent or more, of the load through its condyles.

The following examples further illustrate the invention:

### Example 1

CPT-coated MgTTZ ceramic specimens were prepared and tested as follows:

Single shear static test specimens were ten ¾-inch (1.905-cm) diameter discs, which were bonded to 1-inch (2.54 cm) long CoCr alloy bars with an epoxy adhesive. The coating was applied to one end of the ceramic discs prior to epoxy bonding of the other side. The coating was a TPS porous coating with CPT sprayed onto the MgTTZ specimens roughened by grit-blasting with 16-grit alumina, for providing what was believed to be a target surface finish of 12-Ra in microns. The CPT powder was plasma sprayed robotically under vacuum with Argon pickup gas with Hydrogen gas enhancer at an about 80-psi (about 5.6-kg/cm²) pressure, with Argon gas cooling from lateral spray jets.

Two sheets of Cytec FM-1000 epoxy adhesive were used.

The tests used a single shear fixture, which applied a pure shear load to the bonded interface without inducing a bending stress, as described in ASTM F-1044-05. An Instron model TTD universal testing machine was used, employing a crosshead speed of 0.060 inch (1.5 mm) per minute.

The following static shear strength results were obtained:

| # | Diameter | | Area | | Max. Load | | Shear Stress | | Failure Mode |
|---|---|---|---|---|---|---|---|---|---|
| 1 | 0.750 | inch | 0.4418 | inch² | 2730 | lbs. | 6179 | psi | A |
| | (1.905 | cm) | (2.850 | cm²) | (1238 | kg) | (434.5 | kg/cm²) | |
| 2 | 0.750 | inch | 0.4418 | inch² | 2890 | lbs. | 6541 | psi | A |
| | (1.905 | cm) | (2.850 | cm²) | (1311 | kg) | (460.0 | kg/cm²) | |
| 3 | 0.748 | inch | 0.4394 | inch² | 2940 | lbs. | 6690 | psi | A |
| | (1.8999 | cm) | (2.835 | cm²) | (1334 | kg) | (470.5 | kg/cm²) | |
| 4 | 0.750 | inch | 0.4418 | inch² | 2870 | lbs. | 6496 | psi | A |
| | (1.905 | cm) | (2.850 | cm²) | (1302 | kg) | (456.8 | kg/cm²) | |
| 5 | 0.750 | inch | 0.4418 | inch² | 2860 | lbs. | 6474 | psi | A |
| | (1.905 | cm) | (2.850 | cm²) | (1297 | kg) | (455.2 | kg/cm²) | |
| 6 | 0.750 | inch | 0.4418 | inch² | 3180 | lbs. | 7198 | psi | 70% A 30% G |
| | (1.905 | cm) | (2.850 | cm²) | (1442 | kg) | (506.2 | kg/cm²) | |
| 7 | 0.750 | inch | 0.4418 | inch² | 3140 | lbs. | 7107 | psi | 30% A 70% G |
| | (1.905 | cm) | (2.850 | cm²) | (1424 | kg) | (499.8 | kg/cm²) | |
| 8 | 0.750 | inch | 0.4418 | inch² | 3000 | lbs. | 6790 | psi | A |
| | (1.905 | cm) | (2.850 | cm²) | (1361 | kg) | (477.5 | kg/cm²) | |
| 9 | 0.750 | inch | 0.4418 | inch² | 3070 | lbs. | 6949 | psi | A |
| | (1.905 | cm) | (2.850 | cm²) | (1392 | kg) | (488.7 | kg/cm²) | |
| 10 | 0.749 | inch | 0.4408 | inch² | 2970 | lbs. | 6741 | psi | 10% A 90% G |
| | (1.9025 | cm) | (2.844 | cm²) | (1347 | kg) | (474.0 | kg/cm²) | |

A = Adhesion failure between the ceramic and TPS coating.
G = Glue failure between the TPS coating and the make-up part.

Thus, the average static shear strength for these ten specimens was 6716 psi (472.3 kg/cm²). Compare, FIG. 53.

### Example 2

Two CPT TPS-coated MgTTZ tensile coupons were also prepared essentially as set forth in Example 1 as coupon specimens, and tested. The results were 7,466 psi (525.0 kg/cm²) and 7,683 psi (540.3 kg/cm²).

### Example 3

Shear fatigue test specimens were also prepared essentially as set forth in Example 1 as nine ¾-inch (1.905-cm) diameter ceramic bars, with each bar having an approximately 1-inch (2.54-cm) length. The TPS coating was applied to one end of each ceramic bar, which was bonded to a mating metal cylinder to the coated end of each test sample using an epoxy adhesive. Two sheets of Cytec FM-1000 epoxy adhesive were used.

The tests used a single shear fixture as shown in ASTM F-1160-98, which applied a pure shear load to the bonded interface without inducing a bending stress. An Instron model TTD universal testing machine was used, employing a crosshead speed of 0.100 inch (2.54 mm) per minute. The fatigue tests were done on either a 2000-lb. (9072-kg) capacity Baldwin/Sonntag axial fatigue machine or a 5000-lb. (2268-kg) capacity Krouse axial fatigue machine, depending on the fatigue loads. The testing speed was either twenty-five or thirty cycles per second, depending on the machine employed. The test systems were in current calibration. The load readout system was calibrated to ANSI/NCSL Z540-1 and ISO 10012-1:1992(E). The following shear fatigue test data results were obtained:

| | | | | | Max.-Min. | | Maximum | | Cycles | Failure |
|---|---|---|---|---|---|---|---|---|---|---|
| # | Diameter | | Area | | Loads | | Stress | | to Failure | Mode |
| 1 | 0.751 | inch | 0.4430 | inch² | 1772-177 | lbs. | 4000 | psi | 10,000,000 | N |
| | (1.9075 | cm) | (2.858 | cm²) | (803.8-80.3 | kg) | (281 | kg/cm²) | | |
| 2 | 0.749 | inch | 0.4406 | inch² | 2203-220 | lbs. | 5000 | psi | 86,400 | B |
| | (1.9025 | cm) | (2.843 | cm²) | (999.3-99.8 | kg) | (352 | kg/cm²) | | |
| 3 | 0.751 | inch | 0.4430 | inch² | 2215-222 | lbs. | 5000 | psi | 54,300 | G |
| | (1.9075 | cm) | (2.858 | cm²) | (1005-101 | kg) | (352 | kg/cm²) | | |
| 4 | 0.751 | inch | 0.4430 | inch² | 1772-177 | lbs. | 4000 | psi | 2,809,000 | B + G |
| | (1.9075 | cm) | (2.858 | cm²) | (803.8-80.3 | kg) | (281 | kg/cm²) | | |
| 5 | 0.749 | inch | 0.4406 | inch² | 1542-154 | lbs. | 3500 | psi | 10,000,000 | N |
| | (1.9025 | cm) | (2.843 | cm²) | (699.4-69.9 | kg) | (246 | kg/cm²) | | |
| 6 | 0.751 | inch | 0.4430 | inch² | 1550-155 | lbs. | 3500 | psi | 10,000,000 | N |
| | (1.9075 | cm) | (2.858 | cm²) | (703.1-70.3 | kg) | (246 | kg/cm²) | | |
| 7 | 0.749 | inch | 0.4406 | inch² | 1980-198 | lbs. | 4500 | psi | 371,000 | B |
| | (1.9025 | cm) | (2.843 | cm²) | (898.1-89.8 | kg) | (316 | kg/cm²) | | |
| 8 | 0.750 | inch | 0.4418 | inch² | 1546-155 | lbs. | 4500 | psi | 10,000,000 | N |
| | (1.905 | cm) | (2.850 | cm²) | (701.2-870.3 | kg) | (316 | kg/cm²) | | |
| 9 | 0.749 | inch | 0.4406 | inch² | 1983-198 | lbs. | 4500 | psi | 54,200 | G |
| | (1.9025 | cm) | (2.843 | cm²) | (899.5-89.8 | kg) | (316 | kg/cm²) | | |

B = Failure between the ceramic substrate and the TPS coating.
G = Glue failure between the TPS coating and the metal make-up plug.
B + G = 20% B-type failure with 80% G-type failure.
N = No failure - test stopped at 10,000,000 cycles.

### Example 4

Five additional CPT TPS-coated MgTTZ buttons (discs) were prepared essentially as set forth in Example 1, and tested (ASTM F-1044-05). The results are tabulated as follows:

| | Coating | | | | | | Failure |
|---|---|---|---|---|---|---|---|
| # | Thickness | | Ceramic Ra | | Stress | | Mode |
| 1 | 0.018 | inch | 266 | microinches | 9605 | psi | A |
| | (0.46 | mm) | (6.76 | microns) | (675.5 | kg/cm²) | |
| 2 | 0.020 | inch | 272 | microinches | 8152 | psi | A |
| | (0.51 | mm) | (6.91 | microns) | (573.3 | kg/cm²) | |
| 3 | 0.018 | inch | 266 | microinches | 9554 | psi | 50% A |
| | (0.46 | mm) | (6.76 | microns) | (671.9 | kg/cm²) | 50% G |
| 4 | 0.018 | inch | 282 | microinches | 11242 | psi | 40% A |
| | (0.46 | mm) | (7.16 | microns) | (790.58 | kg/cm²) | 60% G |
| 5 | 0.017 | inch | 268 | microinches | 8777 | psi | G |
| | 0.43 | mm) | (6.81 | microns) | (617.2 | kg/cm²) | |

A = Adhesion failure between the ceramic and TPS coating.
G = Glue failure between the TPS coating and the make-up part.
Ra = surface finish, an arithmetical mean roughness of the surface.

The average shear stress strength for these samples thus was 9426 psi (662.9 kg/cm²).

### Example 5

A number of CPT TPS-coated MgTTZ cruciate-retaining femoral components for a human knee joint implant are prepared by TPS onto the inner “box” surfaces of the ceramic body to provide a surface finish in microns of about from 6-Ra to 8-Ra or about from 11-Ra to 13-Ra, including of a 12-Ra. A 16-grit aluminum oxide can be sprayed at an 80-psi (a 5.6-kg/cm²) pressure to grit blast the MgTTZ. The CPT porous coating can be about from 0.015 to 0.025 of an inch (about from 0.038 to 0.064 cm) thick with an about 100-micron to about 300-micron pore size. Extraordinary shear and fatigue stress strength may be encountered. On some samples, the coating does not hold to the ceramic nearly as well.

### Example 6

A number of CPT TPS-coated MgTTZ buttons or femoral components for a human knee joint implant are prepared by TPS onto surfaces of the ceramic samples. The ceramic surfaces are not grit blasted. Target surfaces that are polished and coated by overspray, and that are not polished, i.e., naturally rough from manufacture such as on a tibial tray liner, receive and accept the coating. Extraordinary shear and fatigue stress strength may be encountered. On some samples, the coating does not hold to the ceramic nearly as well.

### CONCLUSION TO THE INVENTION

The present invention is thus provided. Various feature(s), part(s), step(s), subcombination(s) and/or combination(s) can be practiced with or without reference to other feature(s), part(s), step(s), subcombination(s) and/or combination(s) in the practice of the invention, and numerous adaptations and modifications can be effected within its spirit, the literal claim scope of which is particularly pointed out as follows:

What is claimed is:

1. A coated ceramic comprising a fired ceramic body having a receiving surface that can serve as a substrate for a metal or matal alloy coating; and, on at least part of the receiving surface, the metal or metal alloy coating, wherein:
the fired ceramic body is a zirconia ceramic;
the metal or metal alloy coating is from a metal or metal alloy other than by tantalum vapor deposition; and
the coated ceramic has a static shear strength between the receiving surface of the ceramic body and the metal or metal alloy coating thereon of at least about 2,000 pounds.

2. The coated ceramic of claim 1, wherein the receiving surface of the fired ceramic body is MgTTZ.

3. The coated ceramic of claim 1, wherein the metal or metal alloy coating includes titanium.

4. The coated ceramic of claim 2, wherein the metal or metal alloy coating includes titanium.

5. The coated ceramic of claim 4, wherein the metal or metal alloy coating is CPT.

6. The coated ceramic of claim 5, wherein the CPT is a porous coating.

7. The coated ceramic of claim 1, wherein said static shear strength is at least about 3,000 pounds.

8. The coated ceramic of claim 7, wherein said static shear strength is at least about 5,000 pounds.

9. The coated ceramic of claim 1, which comprises a prosthetic surgical load bearing implant.

10. The coated ceramic of claim 2, which comprises a prosthetic surgical load bearing implant.

11. The coated ceramic of claim 5, which comprises a prosthetic surgical load bearing implant.

12. The coated ceramic of claim 6, which comprises a prosthetic surgical load bearing implant.

13. The coated ceramic of claim 7, which comprises a prosthetic surgical load bearing implant.

14. The coated ceramic of claim 8, which comprises a prosthetic surgical load bearing implant.

15. The coated ceramic of claim 6, which includes an articular surface and is selected from the group consisting of a femoral component for a knee joint, a tibial component for a knee joint, a patellofemoral replacement, a unicompartmental femoral component for a knee joint, a cup for an enarthrodial joint, a talus cap for an ankle joint, a femoral component for an ankle joint, a great toe implant, and a cap for a temporal mandibular joint.

16. The coated ceramic of claim 15, which is a femoral and/or tibial component for a non-rotating hinge knee.

17. The coated ceramic of claim 15, which is a femoral and/or tibial component for a rotating hinge knee.

18. The coated ceramic of claim 15, wherein said static shear strength is at least about 3,000 pounds.

19. The coated ceramic of claim 18, wherein said static shear strength is at least about 5,000 pounds.

20. The coated ceramic of claim 19, wherein said static shear strength is at least about 7,000 pounds.
